(19) United States
(12) Patent Application Publication
(10) Pub. No.: US 2020/0181591 A1
(43) Pub. Date: Jun. 11, 2020

(54) COMPOSITIONS AND METHODS FOR SITE-DIRECTED DNA NICKING AND CLEAVING

(71) Applicant: Gen9, Inc., Boston, MA (US)

(72) Inventors: Joseph Jacobson, Newton, MA (US); Ishtiaq Saaem, Chelsea, MA (US); Michael E. Hudson, Framingham, MA (US); Devin Leake, Lexington, MA (US)

(73) Assignee: Gen9, Inc., Boston, MA (US)

(21) Appl. No.: 16/713,529

(22) Filed: Dec. 13, 2019

Related U.S. Application Data
(63) Continuation of application No. 15/324,522, filed on Jan. 6, 2017, now abandoned.
(60) Provisional application No. 62/065,238, filed on Oct. 17, 2014.
(60) Provisional application No. 62/022,617, filed on Jul. 9, 2014.

(51) Int. Cl. C12N 9/22; C12N 15/62; C12N 15/66; C12N 15/10; C12P 19/34

(52) U.S. Cl. CPC C12N 9/22; C12N 15/62; C07K 2319/00; C12N 15/102; C12P 19/34; C12N 15/66

(57) ABSTRACT

Aspects of the disclosure related to compositions and methods for site-directed DNA nicking and/or cleaving, and use thereof in, for example, polynucleotide assembly.

### CROSS REFERENCE TO RELATED APPLICATIONS

This application is a continuation under 35 U.S.C. § 120 of U.S. application Ser. No. 15/324,522, filed on Jan. 6, 2017, which is a national stage filing under 35 U.S.C. § 371 of PCT International Application No. PCT/US2015/039517, filed Jul. 8, 2015, which claims the benefit under 35 U.S.C. § 119(e) of U.S. Provisional Application Nos. 62/022,617 filed Jul. 9, 2014 and 62/065,238 filed Oct. 17, 2014. The entire content of each of the above referenced applications is incorporated herein by reference in their entirety.

### REFERENCE TO A SEQUENCE LISTING

This specification includes a sequence listing, submitted herewith, which includes the file entitled “G091970009US03-SEQ-ROS.txt” having the following size: 85,587 bytes which was created on Dec. 13, 2019, the content of which is incorporated by reference herein.

### FIELD

The present disclosure relates to compositions and methods for site-directed DNA nicking and cleaving, useful in, for example, in vitro nucleic acid assembly.

### BACKGROUND

Recombinant and synthetic nucleic acids have many applications in research, industry, agriculture, and medicine. Recombinant and synthetic nucleic acids can be used to express and obtain large amounts of polypeptides, including enzymes, antibodies, growth factors, receptors, and other polypeptides that may be used for a variety of medical, industrial, or agricultural purposes. Recombinant and synthetic nucleic acids can also be used to produce genetically modified organisms including modified bacteria, yeast, mammals, plants, and other organisms. Genetically modified organisms may be used in research (e.g., as animal models of disease, as tools for understanding biological processes, etc.), in industry (e.g., as host organisms for protein expression, as bioreactors for generating industrial products, as tools for environmental remediation, for isolating or modifying natural compounds with industrial applications, etc.), in agriculture (e.g., modified crops with increased yield or increased resistance to disease or environmental stress, etc.), and for other applications. Recombinant and synthetic nucleic acids may also be used as therapeutic compositions (e.g., for modifying gene expression, for gene therapy, etc.) or as diagnostic tools (e.g., as probes for disease conditions, etc.).

Numerous techniques have been developed for modifying existing nucleic acids (e.g., naturally occurring nucleic acids) to generate recombinant nucleic acids. For example, combinations of nucleic acid amplification, mutagenesis, nuclease digestion, ligation, cloning and other techniques may be used to produce many different recombinant nucleic acids. Chemically synthesized polynucleotides are often used as primers or adaptors for nucleic acid amplification, mutagenesis, and cloning.

Techniques also are being developed for de novo nucleic acid assembly whereby nucleic acids are made (e.g., chemically synthesized) and assembled to produce longer target nucleic acids of interest. For example, different multiplex assembly techniques are being developed for assembling oligonucleotides into larger synthetic nucleic acids that can be used in research, industry, agriculture, and/or medicine. However, one limitation of current assembly techniques is the unsatisfactory tools available to efficiently produce precisely designed synthetic oligonucleotides that are the building blocks to be assembled into desired nucleic acids. Rather, common techniques such as cleaving with restriction enzymes require introduction of specific recognition sites and upon re-ligation of the cleavage products, often leave behind extraneous nucleotide bases that are undesirable. Even where type IIS restriction enzymes are used which cut outside of the recognition site, it is still necessary to engineer the corresponding recognition sites into the construction oligonucleotides.

Thus, a need exists for an efficient DNA editing tool that can produce precisely designed synthetic oligonucleotides, to assist high-throughput DNA synthesis and assembly.

### SUMMARY

Compositions and methods for site-directed DNA nicking and cleaving are disclosed herein. In addition, methods for DNA assembly are described.

In one aspect, the disclosure provides fusion proteins comprising a catalytically inactive Cas9 fused directly or indirectly to the catalytic domain of a nuclease. The catalytic domain may be, for example, the cleavage or cleaving domain of an endonuclease. In some embodiments, the endonuclease may be a restriction endonuclease, including, for example a type IIS restriction endonuclease. Embodiments include endonucleases that are wild type and/or catalytically active in a dimeric or multimeric form, including, without limitation, FokI, BsaI, AlwI, and BfilI. According to aspects of the disclosure, the nuclease catalytic domain of the fusion proteins of the disclosure may include a mutation that modifies the cleavage activity. For example, a catalytic domain of the endonuclease may include a modification that renders the nuclease catalytic domain a nickase that cleaves only one strand of a double-stranded oligonucleotide. In embodiments of the disclosure in which the catalytic domain functions in a dimeric or multimeric form, the catalytic domain may include a mutation on fewer than all of the monomers that make up the dimer or multimers, and/or two or more monomers may include different mutations. For example, FokI cleavage requires dimerization of the catalytic domain and thus, a wild-type monomer and a mutated, catalytically inactive monomer can dimerize to form a nickase. In certain embodiments, the nickase or nuclease activity may be provided by a hybrid between two or more different endonucleases and/or their catalytic domains. In one non-limiting example, the hybrid is FokI/BsaI.

Aspects of the disclosure relate to compositions and systems for site-directed nicking and cleaving of synthetic oligonucleotides, comprising: (a) a fusion protein comprising a Cas9 bound or fused, directly or indirectly, to one or more monomers of a dimeric or multimeric catalytic domains of a nuclease (“fCas9”); and (b) a second or more such monomers that are not bound to the same Cas9 as the bound monomers of (a). Such second monomer may be bound to another protein (including, for example, a second Cas9) or unbound. According to an embodiment of the disclosure, such compositions and systems may further comprise one or more gRNAs having a designed sequence (e.g., non-naturally occurring) bound to the Cas9; and may further comprise one or more designed oligonucleotides having a recognition region (e.g., non-naturally occurring) that is complementary to the gRNA sequence. As a result, the gRNA:fCas9 complex can specifically bind to the oligonucleotides at the recognition region, directing the catalytic domain of the nuclease to cleave or nick at a predetermined distance from the binding site. In some embodiments, a plurality (e.g., 3, 4, 5, 10, 20, 50, or more or less) of oligonucleotides each having a recognition region (e.g., non-naturally occurring) that is complementary to or the same as the gRNA, wherein the plurality of oligonucleotides (e.g., excluding the recognition region) together comprise a target polynucleotide. According to one embodiment, each of the plurality of oligonucleotides may comprise a flanking region on the 3′ terminus, 5′ terminus, or both termini; such flanking region comprising a primer site and/or a recognition region (e.g., non-naturally occurring) complementary to a gRNA sequence. In one aspect, the primer site may be or include, in whole or in part, the recognition region that is complementary to a gRNA sequence. The plurality of oligonucleotides may together comprise a target polynucleotide with or without the flanking regions.

According to one embodiment, the compositions and systems of the disclosure comprise a first Cas9-nuclease fusion protein that is bound to a first gRNA and a second Cas9-nuclease fusion protein bound to a second gRNAs that is different from the first gRNA. Additional different gRNAs (a third, fourth, fifth, etc. gRNA) may be employed in the compositions and systems of the disclosure. In one aspect, the first and second gRNA sequences comprise non-naturally occurring sequences that are complementary to each other and which may be employed in separate steps of methods of the disclosure. According to another aspect, composition and systems of the disclosure comprise first and second gRNA sequences that are not complementary.

The disclosure also provides methods of using the compositions and systems described herein in applications of synthetic biology. For example, methods for nucleic acid synthesis and assembly using the compositions and systems of the disclosure are disclosed herein. According to some methods of the disclosure, a plurality of oligonucleotides that together comprise a target polynucleotide are provided. Each of the plurality of oligonucleotides comprises a flanking region on one or both termini. The flanking regions comprise a primer site within which is a recognition region comprising a non-naturally occurring sequence. The oligonucleotides may be amplified by a template-driven enzymatic reaction such as PCR. Following amplification, the plurality of oligonucleotides (each comprising a P strand and a complementary N strand) are contacted with a Cas9-nuclease fusion protein such as a catalytically inactive Cas9 fused to a monomer of a FokI catalytic domain. Bound to the Cas9 is a designed synthetic gRNA that is non-naturally occurring and complementary to the recognition region in the flanking region of the P and/or the N strand of each of the plurality of oligonucleotides. The plurality of oligonucleotides are brought into contact with the Cas9-FokI fusion protein in the presence of a second monomer of the FokI (or another endonuclease) catalytic domain (which can be stand-alone or in another Cas9-nuclease fusion) under conditions suitable for binding of the gRNA to the recognition region of the flanking region of the P strand and dimerization between the FokI monomer of the fusion protein and the second FokI monomer (or the monomer of another endonuclease). In some embodiments, one or both of the FokI monomers are mutated such that only the P or N strand is cut, making the catalytic activity of the FokI dimer that of a nickase. For example, in one embodiment, the FokI monomer of the fusion protein is modified (e.g., mutated, FokI*) such that it does not cut the P strand, but the second FokI monomer cuts the N strand (Cas9-FokI*:FokI or Cas9-FokI*:Cas9-FokI). In another embodiment, the second FokI monomer is mutated such that it does not cut the N strand, but the FokI monomer of the fusion protein cuts the P strand (Cas9-FokI:FokI* or Cas9-FokI:Cas9-FokI*). The different complexes (Cas9-FokI*:FokI, Cas9-FokI*:Cas9-FokI, Cas9-FokI:FokI* and Cas9-FokI:Cas9-FokI*) can be used together in any combination (in one reaction mixture or in a step-wise process) to nick one or both strands of a double-stranded DNA. In another embodiment, both the FokI monomer of the fusion protein and the second FokI monomer are catalytically active such that both the P and N strands are cut and the flanking region is cleaved from the remainder of the oligonucleotide. The resulting oligonucleotides may have blunt ends or sticky ends and may then be ligated in a predefined order to assemble a target polynucleotide, or subjected to further processing such as the production and/or modification of cohesive single-stranded overhanging ends, or polymerase assembly where a polymerase is used to extend the oligonucleotides by one or more nucleotide. According to one embodiment of the disclosure, the one or both flanking regions of the plurality of oligonucleotides are cleaved from the remainder of the oligonucleotides in a first and second nicking steps using different Cas9-nuclease fusion proteins comprising different length linkers, and/or comprising different gRNAs designed to position the catalytic domains of the fusion protein at different locations on the P and N strands so as to produce single-stranded overhanging ends on the oligonucleotides that are designed to permit cohesive end assembly of the oligonucleotides to form a target polynucleotide.

It should be noted that while FokI is used as an exemplary endonuclease to illustrate the present disclosure, one of ordinary skill in the art would understand that other endonuclease can also be used in place of or together with FokI.

In one specific aspect, a method for cleaving a polynucleotide is provided, comprising: (a) nicking a first strand of a double-stranded polynucleotide with a first nickase to produce a first nick, wherein the first nickase is configured to recognize and bind a first site on the double-stranded polynucleotide; and (b) nicking a second strand of the double-stranded polynucleotide with a second nickase to produce a second nick, wherein the second nickase is configured to recognize and bind a second site on the double-stranded polynucleotide, thereby producing a cleaved polynucleotide fragment having an overhang defined by the first nick and the second nick, wherein the overhang is predesigned by selecting the first and second site. The overhang can have a predetermined length and/or sequence such that it can specifically and at least partially anneal with another overhang to facilitate ligation with another oligonucleotide. In some embodiments, the overhang can be 1, 2, 3, 4, 5, 6, 7, 8, 9, 10, 11, 12, 13, 14, 15, 16, 17, 18, 19 or 20 nucleotides in length, or longer.

Another aspect of the disclosure is directed to a composition for site-directed DNA cleavage, comprising: (a) a first nickase bound to a first non-naturally occurring guide sequence such as gRNA, wherein the first nickase is configured to recognize and bind a first site on a double-stranded polynucleotide, and to produce a first nick at a first distance therefrom; and (b) a second nickase bound to a second non-naturally occurring guide sequence such as gRNA, wherein the second nickase is configured to recognize and bind a second site on the double-stranded polynucleotide, and to produce a second nick at a second distance therefrom, wherein the first and second nickase together produces a cleaved polynucleotide fragment having an overhang defined by the first nick and the second nick, wherein the overhang is predesigned by selecting the first and second site.

In some embodiments of the method and composition of the present disclosure, the first nickase or the second nickase each comprises one or more of: Cas9 fused to a nuclease via a linker at the N terminus (“fCas9”), Cas9 fused to a nuclease via a linker at the C terminus (“Cas9f”), RISC complexed with or fused to a nuclease, transcription activator-like effector (TALE) complexed with or fused to a nuclease, zinc-finger complexed with or fused to a nuclease, meganuclease, and any combination thereof. The Cas9 may be catalytically inactive. The nuclease may be incapable of binding to DNA. The nuclease can be any suitable type IIS restriction endonuclease, such as FokI, BsaI, AlwI, and BfilI. In one example, the nuclease is FokI. The FokI may be a catalytically inactive monomer of FokI cleavage domain which may dimerize with a catalytically active monomer of FokI cleavage domain. The FokI can also be a catalytically active monomer of FokI cleavage domain and can dimerize with a catalytically active or inactive monomer of FokI cleavage domain. This way, the first nickase or the second nickase can be a dimer. In some embodiments, the first nickase or the second nickase is a heterodimer. In certain embodiments, in the first nickase, the Cas9 or RISC is directed by a first guide sequence such as gRNA to the first site, wherein the first guide sequence such as gRNA comprises a first sequence that is complementary to the first site. In some embodiments, in the second nickase, the Cas9 or RISC is directed by a second guide sequence such as gRNA to the second site, wherein the second guide sequence such as gRNA comprises a second sequence that is complementary to the second site. The first and second guide sequences, in various embodiments, are non-naturally occurring. In one embodiment, the first nickase and the second nickase nick at a predetermined position upstream or downstream to the first site and the second site, respectively, to produce the first nick and the second nick, respectively. The first and second sites may be selected such that the first nick and the second nick are offset by a predefined number of nucleotides.

A further composition provided by the present disclosure comprises: (a) a first nickase bound to a non-naturally occurring guide sequence such as gRNA, wherein the first nickase is configured to recognize and bind a first site on a double-stranded polynucleotide, and to produce a first nick at a first distance therefrom; and (b) a second nickase configured to recognize and bind a second site on the double-stranded polynucleotide, and to produce a second nick at a second distance therefrom, wherein the first and second nickase together produces a cleaved polynucleotide fragment having an overhang defined by the first nick and the second nick, wherein the overhang is predesigned by selecting the first and second site. In some embodiments, the first nickase comprises one or more of: Cas9 fused to a nuclease via a linker at the N terminus (“fCas9”), Cas9 fused to a nuclease via a linker at the C terminus (“Cas9f”), RISC complexed with or fused to a nuclease, and any combination thereof. The second nickase may comprise one or more of: transcription activator-like effector (TALE) complexed with or fused to a nuclease, zinc-finger complexed with or fused to a nuclease, meganuclease, and any combination thereof.

A method for nucleic acid assembly is also provided, comprising: producing the cleaved polynucleotide fragment according to the above method and/or using the above compositions, and assembling the cleaved polynucleotide fragment with another polynucleotide. In some embodiments, the assembling step comprises ligating the cleaved polynucleotide fragment with another polynucleotide having a complementary overhang to the overhang of the cleaved polynucleotide fragment. The assembling can also comprise polymerase assembly. In various embodiments, the polynucleotide is provided on a solid support, which may be, for example, an array or a bead. The method for nucleic acid assembly may further comprise releasing the ligated product from the solid support.

### BRIEF DESCRIPTION OF THE FIGURES

FIG. 1A illustrates an exemplary fusion protein according to a non-limiting embodiment.

FIG. 1B illustrates an exemplary fusion protein according to a non-limiting embodiment.

FIG. 1C illustrates an exemplary fusion protein according to a non-limiting embodiment.

FIG. 1D illustrates an exemplary fusion protein according to a non-limiting embodiment.

FIG. 2A and FIG. 2B illustrate an exemplary method for the synthesis of a cleaved DNA sequence according to a non-limiting embodiment.

FIG. 3A and FIG. 3B illustrate an exemplary method for the synthesis of a nicked DNA sequence using a mutant FokI-bottom with the fusion protein illustrated in FIG. 1A according to a non-limiting embodiment.

FIG. 3C and FIG. 3D illustrate an exemplary method for the synthesis of a cleaved DNA sequence using a mutant FokI-bottom with the fusion protein illustrated in FIG. 1B according to a non-limiting embodiment.

FIG. 4A and FIG. 4B illustrate an exemplary method for the synthesis of a nicked DNA sequence using a mutant FokI-bottom with the fusion protein illustrated in FIG. 1A according to a non-limiting embodiment.

FIG. 4C and FIG. 4D illustrate an exemplary method for the synthesis of a cleaved DNA sequence using a mutant FokI-top with the fusion protein illustrated in FIG. 1C according to a non-limiting embodiment.

FIG. 5A and FIG. 5B illustrate an exemplary method for the synthesis of a cleaved DNA sequence using the two fusion proteins illustrated in FIG. 1D according to a non-limiting embodiment.

FIG. 6A illustrates an exemplary method for the synthesis of DNA sequences according to a non-limiting embodiment.

FIG. 6B illustrates an exemplary method for the synthesis of extended DNA sequences according to a non-limiting embodiment.

FIG. 7 illustrates an exemplary method of the disclosure for preparing oligonucleotides for assembly into a target polynucleotide comprising cleaving an amplification site from the oligonucleotides in a single cleavage step to produce a blunt double strand terminus.

FIGS. 8A and 8B illustrate an exemplary method of the disclosure for preparing oligonucleotides for assembly into a target polynucleotide comprising cleaving an amplification site from the oligonucleotides in a two nicking steps to produce a single-stranded overhanging terminus.

FIGS. 9A and 9B illustrate an exemplary method of the disclosure for preparing oligonucleotides for assembly into a target polynucleotide comprising cleaving an amplification site from the oligonucleotides in a two nicking steps to produce a single-stranded overhanging terminus.

### DETAILED DESCRIPTION OF THE DISCLOSURE

Aspects of the disclosure relate to compositions and methods for the production of site-directed nicked or cleaved DNA. Aspects of the disclosure further relate to compositions and methods for assembling a polynucleotide from oligonucleotides that have been subject to site-directed nicking or cleaving.

### Definitions

As used herein, “clustered regularly interspaced short palindromic repeats” or “CRISPRs” are DNA loci containing short repetitions of base sequences. CRISPRs play a functional role in phage defense in prokaryotes. Briefly, CRISPRs work as follows. When exposed to a phage infection or invasive genetic element, some members of the bacterial population incorporate short sequences from the foreign DNA (“spacers”) between repeated sequences within the CRISPR locus. The combined unit of repeats and spacers in tandem is referred to as the “CRISPR array.” The CRISPR array is transcribed and then processed into short crRNAs (CRISPR RNAs) each containing a single spacer and flanking repeated sequences. Spacers are derived from foreign DNA (which contains corresponding protospacers that can base pair with the spacers) and are generally stably inherited by daughter cells such that when later exposed to a phage or invasive DNA element with the same sequence, the strain is resistant to infection. CRISPRs are known to operate in conjunction with cognate Cas (CRISPR associated) protein(s) that show specificity to the repeat sequences separating the spacers. The Cas protein(s) operate in conjunction with the crRNA to mediate the cleavage of incoming foreign DNA where the crRNA forms an effector complex with the Cas proteins and guides the complex to the foreign DNA, which is then cleaved by the Cas proteins. There are several pathways of CRISPR activation, one of which requires a tracrRNA (trans-activating crRNA, also transcribed from the CRISPR array) which plays a role in the maturation of crRNA. Then a crRNA/tracrRNA hybrid forms and acts as a guide for the Cas9 to the foreign DNA.

As used herein, “Cas9” (CRISPR associated protein 9) is an RNA-guided DNA nuclease enzyme that can induce site-directed double strand breaks in DNA. In some embodiments, Cas9 can include at least one mutation (e.g., D10A) that renders Cas9 a nickase that nicks a single strand on DNA. In some embodiments, Cas9 can include at least two mutations (e.g., D10A and H840A) that render Cas9 catalytically inactive, and is referred to as “dCas9.”

As used herein, “cleavage” or “cleave” refers to cutting a double stranded DNA, resulting in two DNA molecules having blunt or sticky ends.

The term “complementary” means that two nucleic acid sequences are capable of at least partially base-pairing according to the standard Watson-Crick complementarity rules. For example, two sticky ends can be partially complementary, wherein a region of one overhang complements and anneals with a region or all of the other overhang. The gap(s) can be filled in by chain extension in the presence of a polymerase and single nucleotides, followed by or simultaneously with a ligation reaction.

As used herein, a “dimer” is a macromolecular complex formed by two, non-covalently bound, macromolecules. As used herein, a “homodimer” is formed by two identical molecules. As used herein, a “heterodimer” is formed by two non-identical molecules. In some embodiments, a heterodimer of the present disclosure can be FokI:FokI*, where FokI* contains at least one mutation (e.g., D450A for full-length FokI or D69A for the FokI fragment). The mutation may render FokI catalytically inactive. In some embodiments, one or both of FokI and FokI* can be in the form of a fusion protein where it is fused to, for example, dCas9. In one example, a dimer such as a heterodimer of the present disclosure can be a fusion protein, e.g., dCas9-FokI or dCas9-FokI*, complexed with FokI or FokI*. The dimer of the present disclosure may be an obligate dimer or a non-obligate dimer. As used herein, an “obligate dimer” can be a homodimer or a heterodimer, and can only exist associated to each other and is not found in the monomeric state. A “non-obligate dimer” can be a homodimer or a heterodimer, and can exist in the monomeric state.

As used herein, “FokI” refers to an enzyme naturally found in Flavobacterium okeanokoites. See, for example, Kita et al., “The FokI Restriction-Modification System,” The Journal of Biological Chemistry, Vol. 264, No. 10, pp. 5751-56 (part I) (1989), and Sugisaki, et al., “The FokI Restriction-Modification System,” The Journal of Biological Chemistry, Vol. 264, No. 10, pp. 5757-5761 (part II) (1989), the disclosures of each of which are incorporated by reference herein in its entirety. FokI is a type IIS restriction endonuclease including an N-terminal DNA-binding domain and a non-specific DNA cleavage domain at the C-terminal. Once the protein is bound to duplex DNA via its DNA-binding domain at the recognition site, the DNA cleavage domain is activated and cleaves, without further sequence specificity, the first strand 9 nucleotides downstream and the second strand 13 nucleotides upstream of the nearest nucleotide of the recognition site. In some embodiments, FokI is a full-length protein and is composed of 587 amino acids. In some embodiments, FokI is a partial protein and is composed of less than 587 amino acids. In an embodiment, FokI is a partial protein as in SEQ ID NO.:4. In some embodiments, FokI is wild type. In some embodiments, FokI contains at least one mutation (“FokI*”). In some embodiments, the mutation is D450A. In some embodiments, the mutation is D69A as in the partial FokI sequence of SEQ ID NO.:5.

As used herein, a “fusion protein” or “chimeric protein” is a protein generated through the joining of two or more genes or parts of genes (e.g., fragments) that originally code for two or more separate proteins. In some embodiments, the fusion protein further contains a linker such as XTEN. In some embodiments, a fusion protein includes Cas9 and FokI fused together, optionally via a linker. The Cas9 may be catalytically inactive (e.g., dCas9). The FokI may be catalytically active (e.g., wild type) or catalytically inactive (e.g., containing a mutation (e.g., D450A or D69A)). In some embodiments, the fusion protein binds to a guide RNA. In some embodiments, the fusion protein binds to a specific DNA sequence through, e.g., the guide RNA. In some embodiments, the fusion protein includes a nuclear localization sequence (“NLS”). In some embodiments, the fusion protein binds to FokI and forms a dimer. In some embodiments, the FokI bound to the fusion protein is wild type. In some embodiments, the bound FokI is catalytically inactive. In some embodiments, FokI is full-length. In some embodiments, FokI is a protein fragment.

A “guide sequence” can be any synthetic, non-naturally DNA (double or single stranded), RNA, or other artificial nucleic acid sequence such as peptide nucleic acid (PNA), morpholino and locked nucleic acid (LNA), glycol nucleic acid (GNA) and threose nucleic acid (TNA) that is capable of guiding a protein of interest to a specific sequence by way of complementarity. In certain embodiments, the guide sequence is gRNA.

As used herein, “guide RNA” or “gRNA” represents a synthetic, non-naturally occurring RNA molecule capable of guiding a protein of interest to a specific sequence by way of complementarity. In certain embodiments, the gRNA may be a single hybrid hairpin guide RNA which is a designed to mimic the crRNA:tracrRNA complex to load Cas9 for sequence-specific DNA cleavage or nicking. In some embodiments, gRNA can guide and/or localize a Cas9 protein or a Cas9-nuclease fusion protein to a DNA sequence that is complementary to the gRNA or a partial sequence thereof. In additional embodiments, the gRNA can be a small interfering RNA (siRNA) or microRNA (miRNA), which can bind and direct RISC (RNA-induced silencing complex) to specific sequence of interest.

As used herein, a “linker” is a synthetic (e.g., peptide) sequence or non-peptide moiety that occurs between and physically links two peptide sequences (e.g., protein domains). The peptide sequence can be a full-length protein or a protein fragment, or a peptide. The linker may be positioned between NLS and FokI, and/or between FokI and dCas9. In an embodiment, a linker is used to generate a fusion protein of the present disclosure. In an embodiment, FokI-L8 is used to generate a fusion protein of the present disclosure (e.g., fCas9).

As used herein, “nicking” refers to cutting a single strand (P or N) of a double stranded DNA sequence.

As used herein, a “nickase” is a protein configured to cut a single strand of a double stranded DNA sequence. In some embodiments, a nickase is a fusion protein bound to FokI or FokI* in the form of a heterodimer. In some embodiments, a nickase is a fusion protein bound to an identical nickase, forming a homodimer. In some embodiments, the fusion protein is fCas9 where Cas9 is fused to FokI at the N terminus, optionally via a linker. In some embodiments, the fusion protein is Cas9f where Cas9 is fused to FokI at the C terminus, optionally via a linker. In some embodiments, the Cas9 domain of the fusion protein is catalytically inactive. In some embodiments, the fusion protein contains catalytically active FokI. In some embodiments, the fusion protein contains catalytically inactive FokI. In certain embodiments, the nickase is fCas9 or Cas9f bound to FokI. In some embodiments, the bound FokI is the full-length endonuclease. In some embodiments, the bound FokI is a fragment of the endonuclease. In some embodiments, the bound FokI contains a mutation (e.g., D450A or D69A) that renders the bound FokI catalytically inactive. In some embodiments, the bound FokI is catalytically active. The nickase can also be a TALEN (transcription activator-like effector nuclease), ZFN (zinc-finger nuclease) and/or meganuclease, or a monomer thereof. Exemplary TALENs and ZFNs are reviewed in Joung, et al., “TALENs: a widely applicable technology for targeted genome editing,” Nat. Rev. Mol. Cell Biol. 14, 49-55 (2012) and Urnov, et al., “Genome editing with engineered zinc finger nucleases,” Nat. Rev. Genet. 11, 636-646 (2010), respectively, both incorporated herein by reference in their entirety. Exemplary meganucleases are reviewed in Silva et al., “Meganucleases and Other Tools for Targeted Genome Engineering: Perspectives and Challenges for Gene Therapy,” Curr. Gene Ther. February 2011; 11(1): 11-27, incorporated herein by reference in its entirety.

As used herein, an “oligonucleotide” may be a nucleic acid molecule comprising at least two covalently bonded nucleotide residues. The terms “oligonucleotide”, “polynucleotide” and “nucleic acid” are used interchangeably. In some embodiments, an oligonucleotide may be between 10 and 50,000 nucleotides long. In some embodiments, an oligonucleotide may be between 50 and 10,000 nucleotides long. In some embodiments, an oligonucleotide may be between 100 and 1,000 nucleotides long. For example, an oligonucleotide may be between 10 and 500 nucleotides long, or between 500 and 1,000 nucleotides long. In some embodiments, an oligonucleotide may be between about 20 and about 300 nucleotides long (e.g., from about 30 to 250, 40 to 220, 50 to 200, 60 to 180, or about 65 or about 150 nucleotides long), between about 100 and about 200, between about 200 and about 300 nucleotides, between about 300 and about 400, or between about 400 and about 500 nucleotides long. However, shorter or longer oligonucleotides may be used. An oligonucleotide may be a single-stranded nucleic acid. However, in some embodiments a double-stranded oligonucleotide may be used as described herein. In certain embodiments, an oligonucleotide may be chemically synthesized as described in more detail below. In some embodiments, nucleic acids (e.g., synthetic oligonucleotide) may be amplified before use. The resulting product may be double-stranded. Oligonucleotides can be DNA, RNA and/or other naturally or non-naturally occurring nucleic acids. One or more modified bases (e.g., a nucleotide analog) can be incorporated. Examples of modifications include, but are not limited to, one or more of the following: methylated bases such as cytosine and guanine; universal bases such as nitro indoles, dP and dK, inosine, uracil; halogenated bases such as BrdU; fluorescent labeled bases; non-radioactive labels such as biotin (as a derivative of dT) and digoxigenin (DIG); 2,4-Dinitrophenyl (DNP); radioactive nucleotides; post-coupling modification such as dR-NH2 (deoxyribose-NEb); Acridine (6-chloro-2-methoxiacridine); and spacer phosphoramides which are used during synthesis to add a spacer “arm” into the sequence, such as C3, C8 (octanediol), C9, C12, HEG (hexaethlene glycol) and C18.

As used herein, the term “vector” refers to any genetic element, such as a plasmid, phage, transposon, cosmid, chromosome, artificial chromosome, episome, virus, virion, etc., capable of replication when associated with the proper control elements and which can transfer gene sequences into or between cells. The vector may contain a selection module suitable for use in the identification of transformed or transfected cells. For example, selection modules may provide antibiotic resistant, fluorescent, enzymatic, as well as other traits. As a second example, selection modules may complement auxotrophic deficiencies or supply critical nutrients not in the culture media.

“A plurality” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer in between.

As used herein, the term “about” means within 20%, more preferably within 10% and most preferably within 5%. The term “substantially” means more than 50%, preferably more than 80%, and most preferably more than 90% or 95%.

Other terms used in the fields of recombinant nucleic acid technology, synthetic biology, and molecular biology as used herein will be generally understood by one of ordinary skill in the applicable arts.

### Synthetic Oligonucleotides

Typically, oligonucleotide synthesis involves a number of chemical steps that are performed in a cycle repetitive manner throughout the synthesis with each cycle adding one nucleotide to the growing oligonucleotide chain. The chemical steps involved in a cycle are a deprotection step that liberates a functional group for further chain elongation, a coupling step that incorporates a nucleotide into the oligonucleotide to be synthesized, and other steps as required by the particular chemistry used in the oligonucleotide synthesis, such as e.g. an oxidation step required with the phosphoramidite chemistry. Optionally, a capping step that blocks those functional groups which were not elongated in the coupling step can be inserted in the cycle. The nucleotide can be added to the 5′-hydroxyl group of the terminal nucleotide, in the case in which the oligonucleotide synthesis is conducted in a 3′→5′ direction or at the 3′-hydroxyl group of the terminal nucleotide in the case in which the oligonucleotide synthesis is conducted in a 5′→3′ direction.

For clarity, the two complementary strands of a double stranded nucleic acid are referred to herein as the positive (P) and negative (N) strands. This designation is not intended to imply that the strands are sense and anti-sense strands of a coding sequence. They refer only to the two complementary strands of a nucleic acid (e.g., a target nucleic acid, an intermediate nucleic acid fragment, etc.) regardless of the sequence or function of the nucleic acid. Accordingly, in some embodiments the P strand may be a sense strand of a coding sequence, whereas in other embodiments the P strand may be an anti-sense strand of a coding sequence. It should be appreciated that the reference to complementary nucleic acids or complementary nucleic acid regions herein refers to nucleic acids or regions thereof that have sequences which are reverse complements of each other so that they can hybridize in an antiparallel fashion typical of natural DNA.

In some aspects of the disclosure, the oligonucleotides synthesized or otherwise prepared according to the methods described herein can be used as building blocks for the assembly of a target polynucleotide of interest.

Oligonucleotides may be synthesized on solid support. As used herein, the term “solid support”, “support” and “substrate” are used interchangeably and refers to a porous or non-porous solvent insoluble material on which polymers such as nucleic acids are synthesized or immobilized. As used herein “porous” means that the material contains pores having substantially uniform diameters (for example in the nm range). Porous materials can include but are not limited to, paper, synthetic filters and the like. In such porous materials, the reaction may take place within the pores. The support can have any one of a number of shapes, such as pin, strip, plate, disk, rod, bends, cylindrical structure, particle, including bead, nanoparticle and the like. The support can have variable widths.

The support can be hydrophilic or capable of being rendered hydrophilic. The support can include inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass, magnetic controlled pore glass, ceramics, metals, and the like; either used by themselves or in conjunction with other materials.

In some embodiments, pluralities of different single-stranded oligonucleotides are immobilized at different features of a solid support. In some embodiments, the support-bound oligonucleotides may be attached through their 5′ end or their 3′ end. In some embodiments, the support-bound oligonucleotides may be immobilized on the support via a nucleotide sequence (e.g. degenerate binding sequence), linker (e.g. photocleavable linker or chemical linker). It should be appreciated that by 3′ end, it is meant the sequence downstream to the 5′ end and by 5′ end it is meant the sequence upstream to the 3′ end. For example, an oligonucleotide may be immobilized on the support via a nucleotide sequence or linker that is not involved in subsequent reactions.

Certain embodiments of the disclosure may make use of a solid support comprised of an inert substrate and a porous reaction layer. The porous reaction layer can provide a chemical functionality for the immobilization of pre-synthesized oligonucleotides or for the synthesis of oligonucleotides. In some embodiments, the surface of the array can be treated or coated with a material comprising suitable reactive group for the immobilization or covalent attachment of nucleic acids. Any material, known in the art, having suitable reactive groups for the immobilization or in situ synthesis of oligonucleotides can be used.

In some embodiments, the porous reaction layer can be treated so as to comprise hydroxyl reactive groups. For example, the porous reaction layer can comprise sucrose.

According to some aspects of the disclosure, oligonucleotides terminated with a 3′ phosphoryl group oligonucleotides can be synthesized a 3′→5′ direction on a solid support having a chemical phosphorylation reagent attached to the solid support. In some embodiments, the phosphorylation reagent can be coupled to the porous layer before synthesis of the oligonucleotides. In an exemplary embodiment, the phosphorylation reagent can be coupled to the sucrose. For example, the phosphorylation reagent can be 2-[2-(4,4′-Dimethoxytrityloxy)ethylsulfonyl]ethyl-(2-cyanoethyl)-(N,N-diisopropyl)-phosphoramidite. In some embodiments, the 3′ phosphorylated oligonucleotide can be released from the solid support and undergo subsequent modifications according to the methods described herein. In some embodiments, the 3′ phosphorylated oligonucleotide can be released from the solid support using ammonium hydroxide.

In some embodiments, synthetic oligonucleotides for the assembly may be designed (e.g. sequence, size, and number). Synthetic oligonucleotides can be generated using standard DNA synthesis chemistry (e.g. phosphoramidite method). Synthetic oligonucleotides may be synthesized on a solid support, such as for example a microarray, using any appropriate technique as described in more detail herein. Oligonucleotides can be eluted from the microarray prior to be subjected to amplification or can be amplified on the microarray. It should be appreciated that different oligonucleotides may be designed to have different lengths.

In some embodiments, oligonucleotides are synthesized (e.g., on an array format) as described in U.S. Pat. No. 7,563,600, U.S. patent application Ser. No. 13/592,827, and PCT/US2013/047370 published as WO 2014/004393, which are hereby incorporated by reference in their entireties. For example, single-stranded oligonucleotides are synthesized in situ on a common support wherein each oligonucleotide is synthesized on a separate or discrete feature (or spot) on the substrate. In some embodiments, single-stranded oligonucleotides are bound to the surface of the support or feature. As used herein, the term “array” refers to an arrangement of discrete features for storing, routing, amplifying and releasing oligonucleotides or complementary oligonucleotides for further reactions. In an embodiment, the support or array is addressable: the support includes two or more discrete addressable features at a particular predetermined location (i.e., an “address”) on the support. Therefore, each oligonucleotide molecule of the array is localized to a known and defined location on the support. The sequence of each oligonucleotide can be determined from its position on the support. Moreover, addressable supports or arrays enable the direct control of individual isolated volumes such as droplets. The size of the defined feature can be chosen to allow formation of a microvolume droplet on the feature, each droplet being kept separate from each other. As described herein, features are typically, but need not be, separated by interfeature spaces to ensure that droplets between two adjacent features do not merge. Interfeatures will typically not carry any oligonucleotide on their surface and will correspond to inert space. In some embodiments, features and interfeatures may differ in their hydrophilicity or hydrophobicity properties.

In various embodiments, the synthetic single-stranded or double-stranded oligonucleotides can be non-naturally occurring, e.g., being unmethylated or modified in a way (e.g., chemically or biochemically modified in vitro) such that they become hemi-methylated (only one strand is methylated) or semi-methylated (only a portion of the normal methylation sites are methylated on one or both strands) or hypomethylated (more than the normal methylation sites are methylated on one or both strands), or have non-naturally occurring methylation patterns (some of the normal methylation sites are methylated on one or both strands and/or normally unmethylated sites are methylated). In contrast, naturally-occurring DNA typically contains epigenetic modifications such as methylation at, e.g., the C-5 position of the cytosine ring of DNA by DNA methyltransferases (DNMTs) in vivo. DNA methylation is reviewed by Jin et al., Genes & Cancer 2011 June; 2(6): 607-617, which is incorporated herein by reference in its entirety.

### Site-Directed Nicking and Cleaving

In some embodiments, the disclosure provides compositions and methods for site-directed nicking and/or cleaving. One exemplary composition includes a fusion protein comprising a catalytically inactive Cas9 fused directly or indirectly to the catalytic domain of a nuclease. The nuclease catalytic domain may be, for example, the cleaving domain of an endonuclease. In some aspects, the endonuclease may be a restriction endonuclease, including, for example a type IIS restriction endonuclease. Embodiments include endonucleases that are catalytically active in a dimeric or multimeric form, including, without limitation, FokI, AlwI, and BfilI. The nuclease catalytic domain may include a mutation that modifies the cleavage activity. For example, a catalytic domain may include a modification that renders the nuclease catalytic domain a nickase, e.g., one that cleaves only one strand of a double stranded oligonucleotide. In embodiments where the catalytic domain functions in a dimeric or multimeric form, the catalytic domain may include a mutation on fewer than all of the monomers that make up the dimer or multimers, and/or two or more monomers may include different mutations.

As shown in FIGS. 7-9, aspects of the disclosure relate to compositions and systems for site-directed nicking and cleaving of synthetic oligonucleotides, comprising (a) a fusion protein comprising a Cas9 linked, directly or indirectly, to one or more monomers of a dimeric or multimeric catalytic domains of a nuclease; and (b) a second or more such monomers that are not linked to the same Cas9 as the Cas9-linked monomers. Such second monomer may be linked to another protein (including, for example, a second Cas9) or stand-alone. Components (a) and (b) can bind or complex with each other, forming a dimer (homodimer or heterodimer) that has nuclease activity. According to an embodiment of the disclosure, such compositions and systems may further comprise one or more gRNAs bound to the Cas9; and may further comprise one or more oligonucleotides having a region that is complementary to the gRNA sequence or a part thereof. The gRNA sequence may be naturally occurring or non-naturally occurring and designed to be complementary to a portion of a target oligonucleotide to be nicked or cleaved. The dimer:gRNA complex, by way of annealing of the gRNA to the target oligonucleotide, can bind thereto and exercise its nuclease activity.

In some embodiments, a plurality of oligonucleotides each having a region that is complementary to or is the same as the gRNA can be included, wherein the plurality of oligonucleotides together comprise a target polynucleotide to be assembled from the plurality of oligonucleotides. According to one embodiment, each of the plurality of oligonucleotides can have a flanking region on the 3′ terminus, 5′ terminus, or both termini. The flanking region can include a primer site for PCR amplification and/or a recognition region complementary to the gRNA sequence. The primer site may be or include, in whole or in part, the recognition sequence. The plurality of oligonucleotides may together comprise the target polynucleotide with or without the flanking regions.

It should be noted that one or more primers used herein can be methylated such that the amplified product can be digested with a methylation-sensitive nuclease such as MspJI, SgeI and FspEI. Such nuclease shares both type IIM and type IIS properties; thus, it only recognizes the methylation-specific 4-bp sites, ^{m}CNNR (N=A or T or C or G; R=A or G), and cuts DNA outside of this recognition sequences. Methylated primers and use thereof are disclosed in Chen et al., Nucleic Acids Research, 2013, Vol. 41, No. 8, e93, which is incorporated herein by reference in its entirety.

According to one embodiment, a composition comprising a first Cas9-nuclease fusion protein bound to a first gRNA and a second Cas9-nuclease fusion protein bound to a second gRNA is provided. The first and second gRNAs can be different. In some embodiments, the first and second gRNA sequences are designed to guide the first and second Cas9-nuclease fusion proteins, respectively, to specific positions on a double-stranded DNA sequence, to perform site-directed DNA nicking or cleaving. For example, the first and second fusion proteins can be used to target and nick the P and N strand of the same oligonucleotide, respectively, at predetermined positions, thereby producing a predesigned sticky end. Alternatively, the first and second fusion proteins can be used to target and cleave double strands of different oligonucleotides, thereby producing two or more predesigned sticky ends. Additional different gRNAs (a third, fourth, fifth, etc. gRNA) may be employed to produce nicks at different sites or cuts on more oligonucleotides. In one example, the first and second gRNA sequences comprise sequences that are completely or partially complementary to each other and which may be employed in separate or the same nicking or cleaving step. The first and second gRNA sequences, in some embodiments, are not complementary.

The disclosure also provides methods of using the compositions and systems described herein in applications of, for example, synthetic biology. For example, methods for nucleic acid synthesis and assembly using the compositions and systems disclosed herein are provided. According to one aspect, a plurality of oligonucleotides that together comprise a target polynucleotide are provided. Each of the plurality of oligonucleotides is designed to add a flanking region on one or both termini. The flanking regions can have a primer site completely or partially within, or outside, which is a recognition region for gRNA binding. The oligonucleotides may be amplified by a template-driven enzymatic reaction such as PCR using a primer against the primer site. Following amplification, the plurality of oligonucleotides (each comprising a P strand and a complementary N strand) can be contacted with a Cas9-nuclease fusion protein such as a catalytically inactive Cas9 fused to a first monomer of a type IIS endonuclease (e.g., FokI) catalytic domain. Bound to the Cas9 is a pre-designed, synthetic gRNA complementary to the recognition region of the P and/or the N strand of each of the plurality of oligonucleotides. The plurality of oligonucleotides are brought into contact with the gRNA and the Cas9-nuclease (e.g., Cas9-FokI) fusion protein in the presence of a second monomer of the nuclease (e.g., FokI) catalytic domain under conditions suitable for binding of the gRNA to the recognition region, as well as dimerization between the first nuclease monomer of the Cas9-nuclease fusion protein and the second nuclease monomer. Upon dimerization, the nicking or cleavage activity present in the Cas9-nuclease and/or the second nuclease monomer can act to nick or cleave the target oligonucleotide.

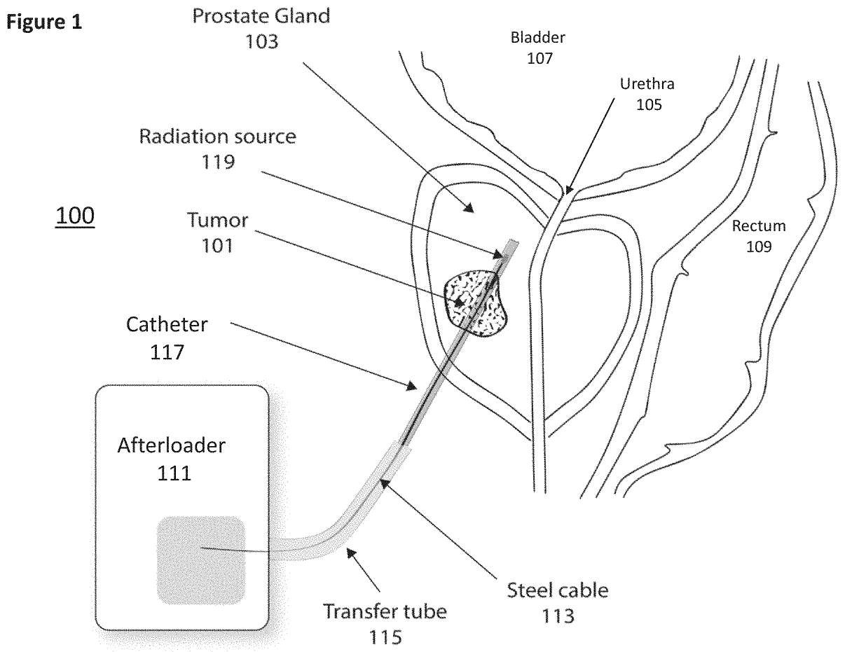

FIGS. 1A-1D depict a few exemplary fusion proteins, fCas9 or Cas9f, for use to nick and/or cleave a double stranded DNA sequence. For example, FIG. 1A illustrates a fusion protein fCas9 including Cas9 (e.g., dCas9) linked, at one terminus (N or C) to a monomer of a catalytic domain of FokI (e.g., wild type) by a linker sequence. The Cas9 can bind to a gRNA which anneals with a complementary sequence DNA at a specific position upstream of FokI. FIG. 1B depicts a fusion protein Cas9f in which Cas9 is linked at the opposite terminus to FokI such that when Cas9 binds a nucleic acid (via complementary gRNA) at a specific position, FokI is placed upstream to Cas9. FIG. 1C illustrates a fusion protein bound to a gRNA, in which Cas9 (e.g., dCas9) is linked by a linker sequence to a monomer of a catalytic domain of FokI that is a catalytically inactive mutant (e.g., D450A). The fusion proteins in FIGS. 1A-1C contain a FokI portion that can dimerize with another FokI monomer (wild type or mutant) to form, for example, a heterodimer. FIG. 1D depicts a fusion protein bound to a gRNA, in which Cas9 (e.g., dCas9) is linked to FokI by a linker sequence. The fusion protein of FIG. 1D is capable of homo-dimerization.

In various embodiments, the gRNA used herein can be designed to contain a sequence that is complementary to the sequence of the desired binding site. As a result, the gRNA can specifically bind to the desired binding site under suitable conditions, directing fCas9 or Cas9f thereto. The FokI portion of fCas9 or Cas9f can then bind, e.g., nonspecifically, to the DNA molecule at a distance from the gRNA binding site. The geometry of fCas9 or Cas9f, e.g., the space between Cas9:gRNA and FokI may determine where FokI binds. The length and/or geometry of the linker can also affect FokI binding. In some embodiments, each specific Cas9-nuclease fusion protein may have a corresponding, specific position where FokI binds. For example, fusion protein 1 may have a FokI binding position that is X1 nucleotides from the gRNA binding site, fusion protein 2 may have a FokI binding position that is X2 nucleotides from the gRNA binding site, and so on. In certain embodiments, the FokI binding position is not fixed; rather, some flexibility (e.g., 1 or 2 or more nucleotides) can be present. For example, for the same fusion protein, FokI may bind at X nucleotides from the gRNA binding site in one reaction, and may bind at X+N (+ indicates downstream) or X−N (− indicates upstream) nucleotides from the gRNA binding site in another reaction, where N is 1 or 2 or 3 or more. After binding, the FokI region of fCas9 or Cas9f can dimerize with a second FokI (e.g., full-length or a fragment, catalytically active or inactive) and can nick or cleave the double stranded nucleic acid. In certain embodiments, FokI nicks or cleaves DNA without binding.

In some embodiments, the fusion protein can further include a nuclear localization sequence (“NLS”) that locates the protein to the nucleus. In certain embodiments, a linker can be used to generate the fusion protein disclosed herein. The linker may be positioned between NLS and FokI, and/or between FokI and dCas9. Table 1 below identifies some non-limiting examples of such linkers. In an embodiment, FokI-L8 is used to generate a fusion protein of the present disclosure (e.g., fCas9).

TABLE 1
Exemplary Linker Sequences

| | NLS-linker- | |
|---|---|---|
| Name | FokI | FokI-linker-dCas9 |
| FokI-(GGS)x3 | GGS | GGSGGSGGS |
| (SEQ ID NO: 13) | | (SEQ ID NO.: 13) |
| FokI-(GGS)x6 | GGS | GGSGGSGGSGGSGGSGGS |
| (SEQ ID NO: 14) | | (SEQ ID NO.: 14) |
| FokI-L0 | GGS | — |
| FokI-L10 | GGS | MKIIEQLPSA |
| (SEQ ID NO.: 15) | | |
| FokI-L2 | GGS | VRHKLKRVGS |
| (SEQ ID NO.: 16) | | |
| FokI-L3 | GGS | VPFLLEPDNINGKTC |
| (SEQ ID NO.: 17) | | |
| FokI-L4 | GGS | GHGTGSTGS GS S |
| (SEQ ID NO.: 18) | | |
| FokI-L5 | GGS | MSRPDPA |
| (SEQ ID NO.: 19) | | |
| FokI-L6 | GGS | GSAGSAAGSGEF |
| (SEQ ID NO.: 20) | | |
| FokI-L7 | GGS | SGSETPGTSESA |
| (SEQ ID NO.: 21) | | |
| FokI-L8 | GGS | SGSETPGTSESATPES |
| (SEQ ID NO.: 22) | | |
| FokI-L9 | GGS | SGSETPGTSESATPEG |
| (SEQ ID NO.: 23) | | GSGGS |
| NLS-(GGS) | GGS | GGSM (SEQ ID NO: 25) |
| NLS-(GGS)x3 | GGSGGSGGS | GGSM (SEQ ID NO: 25) |
| (SEQ ID NO: 13) | (SEQ ID | |
| | NO: 13) | |
| NLS-L1 | VPFLLEPDN | GGSM (SEQ ID NO: 25) |
| | INGKTC | |
| | (SEQ ID | |
| | NO.: 17) | |
| NLS-L2 | GSAGSAAGS | GGSM (SEQ ID NO: 25) |
| | GEF | |
| | (SEQ ID | |
| | NO.: 20) | |
| NLS-L3 | SIVAQLSRP | GGSM (SEQ ID NO: 25) |
| | DPA | |
| | (SEQ ID | |
| | NO.: 24) | |
| Wild-type Cas9 | N/A | N/A |
| Cas9 nickase | N/A | N/A |

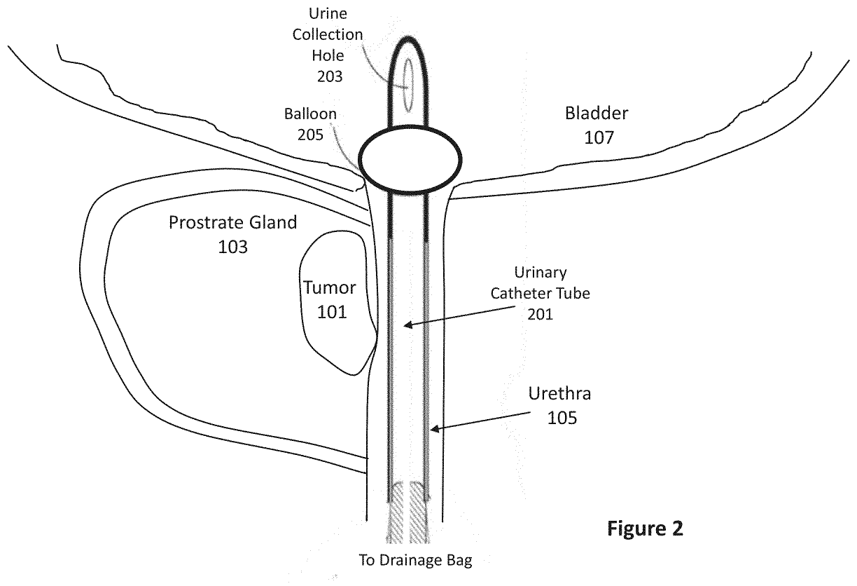

FIGS. 2A-2B depict a method of removing a double stranded “Amp tag” (primer sequence for amplification) from a double stranded sequence. FIG. 2A illustrates a fusion protein including dCas9, FokI, and a linker sequence therebetween (sometimes designated as “fCas9”), bound to at least one gRNA molecule that facilitates site-directed localization of the fusion protein to a specific location on a double stranded molecule comprising, e.g., Amp tag and Sequence A. In an embodiment, the fusion protein is FokI-XTEN-dCas9, as provided in SEQ ID NO.:12 (excluding NLS-GGS). After binding, the FokI region of fCas9 can dimerize with a second, catalytically active FokI and the resulting dimer can cleave the double stranded sequence, producing two double stranded segments as shown in FIG. 2B: the Amp tag and the desired DNA sequence (e.g., Sequence A′) which can be further subject to additional assembly.

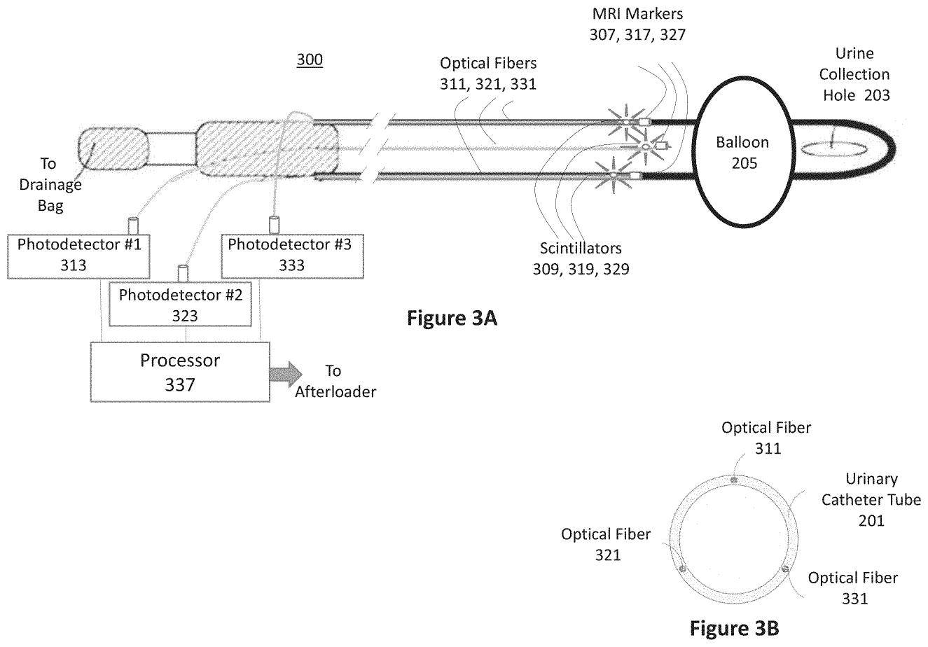

FIGS. 3A-3D illustrate a two-step method of removing an Amp tag from a double stranded sequence. The first step is shown in FIGS. 3A-3B, and the second step is shown in FIGS. 3C-3D. In FIG. 3A, a first fusion protein including dCas9, FokI, and a linker sequence therebetween (“fCas9”), bound to at least one gRNA molecule (e.g., gRNA1), is selectively localized to a specific location, site1, on a double stranded molecule comprising, e.g., Amp tag and Sequence B. gRNA1 contains sequence that is complementary to and anneals with the top strand in the Amp tag at site1. fCas9 is configured such that when bound to the top strand at site1, the FokI portion (“FokI-top”) is positioned downstream to dCas9. In an embodiment, the fusion protein is FokI-XTEN-dCas9 as provided in SEQ ID NO.:12 (excluding NLS-GGS). The FokI-top of fCas9 dimerizes with a second FokI* (e.g., full length or fragment) which contains a mutation, rendering it nuclease-dead (e.g., “FokI (D450A)-bottom” and/or “FokI (D69A)-bottom”). The resulting fCas9:FokI* heterodimer nicks the top strand of the double stranded nucleic acid, producing a nicked molecule having a first nick on the top strand as shown in FIG. 3B. In the second step, a second fusion protein Cas9f:gRNA2, shown in FIG. 3C, is directed to the bottom strand of the nicked molecule at site2, by gRNA2 having a sequence complementary to site2. Cas9f is configured such that when bound to the bottom strand at site2, the FokI-top is positioned upstream to dCas9, while dimerizing with a catalytically inactive FokI* monomer (e.g., “FokI(D450A)-bottom” and/or “FokI (D69A)-bottom”). This Cas9f:FokI* heterodimer produces a second nick on the bottom strand. The net result is a double stranded break, with the Amp tag separated from Sequence B, producing Sequence B′ (FIG. 3D) having a sticky end for further assembly or other manipulation. In some embodiments, site1 and site2 are designed in a way such that the sticky end in Sequence B′ has a desired overhang with a predetermined length and sequence.

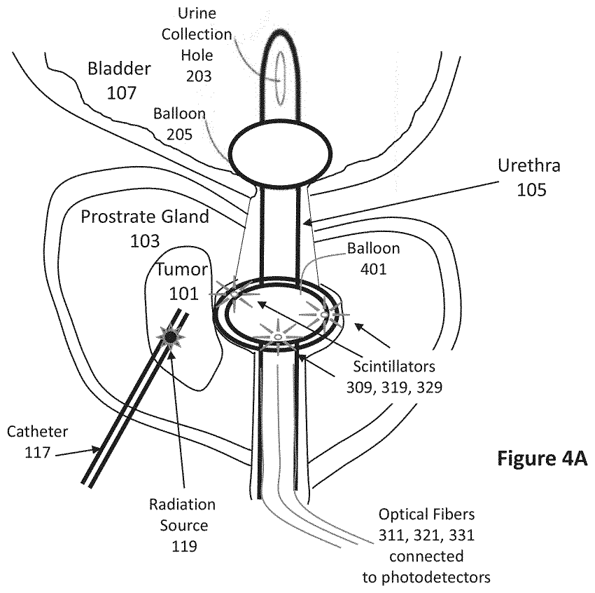

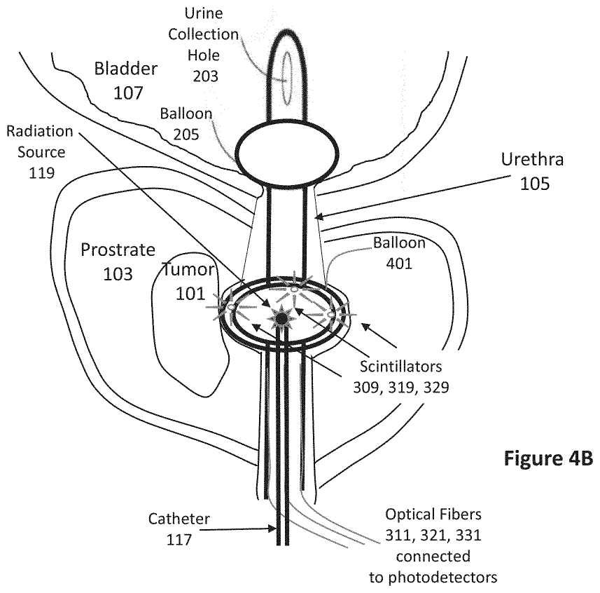

FIGS. 4A-4D illustrate another 2-step method of removing an Amp tag from a double stranded sequence. The first step is shown in FIGS. 4A-4B, and the second step is shown in FIGS. 4C-4D. In FIG. 4A, a first fusion protein including dCas9, FokI, and a linker sequence therebetween (“fCas9”), bound to at least one gRNA molecule (e.g., gRNA1), is selectively localized to a specific location, site1, on a double stranded molecule comprising, e.g., Amp tag and Sequence C. gRNA1 contains sequence that is complementary to and binds with the top strand in the Amp tag at site1. fCas9 is configured such that when bound to the top strand at site1, the FokI portion (“FokI-top”) is downstream to dCas9. In an embodiment, the fusion protein is FokI-XTEN-dCas9 as provided in SEQ ID NO.:12 (excluding NLS-GGS). The FokI-top of fCas9 dimerizes with a second FokI* (e.g., full length or fragment) which contains a mutation, rendering it nuclease-dead (e.g., “FokI (D450A)-bottom” and/or “FokI (D69A)-bottom”). The resulting fCas9:FokI* heterodimer nicks the top strand of the double stranded nucleic acid, producing a nicked molecule having a first nick on the top strand as shown in FIG. 4B. In the second step, a second fusion protein fCas9*:gRNA2, shown in FIG. 4C, is directed to the top strand of the nicked molecule at site2, by gRNA2 having a sequence complementary to site2. fCas9* contains a mutant FokI* portion (e.g., “FokI(D450A)-top” and/or “FokI (D69A)-top”) and is configured such that when bound to the top strand at site2, the FokI*-top is positioned downstream to dCas9, while dimerizing with a catalytically active FokI monomer (FokI-bottom). This fCas9*:FokI heterodimer produces a second nick on the bottom strand. The net result is a double stranded break, with the Amp tag separated from Sequence C, producing Sequence C′ (FIG. 4D) having a sticky end for further assembly or other manipulation. In some embodiments, site1 and site2 are designed in a way such that the sticky end in Sequence C′ has a desired overhang with a predetermined length and sequence.

FIGS. 5A and 5B depict a further method of removing an Amp tag from a double stranded sequence. FIG. 5A illustrates a first and second fusion protein, each including Cas9, FokI, and a linker sequence (“fCas9”), bound to gRNA1 and gRNA2, respectively, which direct selective localization of the first and second fusion proteins to specific locations, site1 and site2, respectively, on a double stranded molecule comprising, e.g., Amp tag and Sequence D. In an embodiment, each of the first and second fusion protein is FokI-XTEN-dCas9, as provided in SEQ ID NO.:12 (excluding NLS-GGS). Site1 and site2 are designed such that when the first and second fusion proteins are localized, the two FokI regions of fCas9 can contact and dimerize with each other to act as a catalytically active endonuclease. As such, the fCas9:fCas9 homodimer can cleave the double stranded molecule, separating the Amp tag and producing Sequence D′ (FIG. 4D) having a sticky end for further assembly or other manipulation.

FIGS. 2A-5B show exemplary methods for generating at least one overhang that can be used for polynucleotide assembly as discussed below (FIGS. 6A and 6B). It should be noted that the overhangs can each be designed to have a predetermined length and/or sequence such that it can specifically and at least partially anneal with another overhang to facilitate ligation with another oligonucleotide. In some embodiments, the overhang can be 1, 2, 3, 4, 5, 6, 7, 8, 9, 10, 11, 12, 13, 14, 15, 16, 17, 18, 19 or 20 nucleotides in length, or longer.

FIGS. 6A-6B illustrate oligonucleotide sequences comprising DNA sequences produced from any of the methods described herein and illustrated in, for example, FIGS. 2A-2B, 3A-3D, 4A-4D and 5A-5B (e.g., Sequences A′, B′, C′, and/or D′) being subject to polynucleotide synthesis and assembly on bead solid support. For illustration purpose only, beads are shown in FIGS. 6A-6B but other solid supports such as a microarray device can also be used. A first plurality of oligonucleotides, A₀, B₀, C₀, . . . and ZZ₀, can each be operably linked to a bead (e.g., each oligonucleotide can have a cleavable linker moiety synthesized or built therein), such that after synthesis, polynucleotides can be cleaved therefrom into a solution. The first plurality of oligonucleotides can be assembled with a second plurality of oligonucleotides, A₁, B₁, C₁, . . . and ZZ₁, each having a sticky end that is completely or partially complementary to that of A₀, B₀, C₀, . . . and ZZ₀, respectively, as shown in FIG. 6A. Assembly can be achieved using any one or more of ligation, primer extension, and PCR. A second assembly step is shown in FIG. 6B, adding a third plurality of oligonucleotides, A₂, B₂, C₂, . . . and ZZ₂. This process can be repeated multiple times until a desired plurality of polynucleotide products are built. In some embodiments, the added oligonucleotides do not have a cleavable linker.

Additional examples of nicking and cleaving are shown in FIGS. 7-9B. In some embodiments, to form a dimer with an endonuclease activity, the first and second FokI monomers can both be wild type. As shown in the embodiment of FIG. 7, both the FokI monomer of the fusion protein and the second FokI monomer are catalytically active such that both the P and N strands are cut and the flanking region is cleaved from the remainder of the oligonucleotide. The oligonucleotides may then be ligated in a predefined order to assemble a target polynucleotide, or subject to further processing such as the production of cohesive single-stranded overhanging ends, or polymerase assembly.

As shown in the embodiments of FIGS. 8A-8B and 9A-9B, one of the two FokI monomers is mutated such that only the P or N strand is cut, making the catalytic activity of the FokI dimer that of a nickase. For example, in one embodiment, the FokI monomer of the fusion protein is modified such that it does not cut the P or top strand, but the second FokI monomer cuts the N or bottom strand (FIG. 9B). In another embodiment, the second FokI monomer is mutated such that it does not cut the N strand, but the FokI monomer of the fusion protein cuts the P strand (FIGS. 8A, 8B and 9A).

FIGS. 8A-8B and 9A-9B show two different designs where the flanking regions of the plurality of oligonucleotides are cleaved from the remainder of the oligonucleotides in two nicking steps. In the first nicking step shown in FIGS. 8A and 9A, the top strand is cut by the top FokI monomer (FokI—top) of the fusion protein which is directed thereto by annealing of the gRNA1, while the bottom strand remains intact due to the inactive, second FokI monomer (FokI*-bottom). The second nicking steps in FIGS. 8B and 9B are different. In FIG. 8B, the bottom strand is cut by the bottom FokI monomer (FokI—bottom) of the fusion protein which is directed thereto by annealing of the gRNA2, without further nicking the top strand due to the inactive, second FokI monomer (FokI*—top). gRNA1 and gRNA2 can be designed to be completely or partially complementary to each other, such that the first nick and the second nick are offset by a pre-selected number (X) of nucleotides. For example, gRNA1 and gRNA2 can be designed to be completely complementary to each other, while the two fusion proteins in FIGS. 8A and 8B are engineered to have different linkers such that they nick at different distance from the gRNA binding position. Alternatively, the two fusion proteins may be identical and cut at the same distance from the gRNA binding site, but gRNA1 and gRNA2 are designed to offset by X nucleotides. In further embodiments, both linker length and gRNA1 and/or gRNA2 sequence can be varied, with the combination of the two strategies resulting in the predesigned overhang of X nucleotides.

Referring now to FIG. 9B, in the second nicking step, the FokI monomer of the fusion protein is inactive and does not cut the top strand, but the second FokI monomer cuts the bottom strand. Here, gRNA1 and gRNA2 can be designed to be completely or partially identical to each other, such that the first nick and the second nick are offset by a pre-selected number (X) of nucleotides. For example, gRNA1 and gRNA2 can be designed to be completely identical, while the two fusion proteins in FIGS. 9A and 9B are engineered to have different linkers such that they nick at different distance from the gRNA binding position. Alternatively, the two fusion proteins may have the same or similar linker and cut at the same distance from the gRNA binding site, but gRNA1 and gRNA2 are designed to offset by X nucleotides. In further embodiments, both linker length and gRNA1 and/or gRNA2 sequence can be varied, with the combination of the two strategies resulting in the predesigned overhang of X nucleotides.

In any of the embodiments of FIGS. 2A-5B and 7-9B, different linkers of different length/geometry in the Cas9-nuclease fusion proteins, and/or different gRNAs designed to position the catalytic domains of the fusion protein at different locations on the P and N strands may be used so as to produce single-stranded overhanging ends that are designed to permit cohesive end ligation and/or polymerase assembly of the construction oligonucleotides to form a target polynucleotide.

The target polynucleotide can be produced in a one-pot reaction where all construction oligonucleotides are mixed and ligated together. Ligation can also be performed sequentially (ligating oligonucleotides one by one) or hierarchically (ligating subpools of the oligonucleotides into one or more subconstructs which are then ligated into the final target construct). It should be noted that one or more of the construction oligonucleotides, one or more of the guide sequences, one or more of the subconstructs, and/or the final target construct can be non-naturally occurring, e.g., being unmethylated or modified in a way (e.g., chemically or biochemically modified in vitro) such that they become hemi-methylated or semi-methylated or hypomethylated, or have non-naturally occurring methylation patterns. Such non-naturally occurring methylation and methylation patterns can be used to regulate, for example, gene expression.

It should be noted that while FIGS. 1-9 illustrate cleavage and assembly of linear oligonucleotides, circular materials such as plasmids can also be subject to similar cleavage and assembly steps. For example, genes or fragments thereof can be first cloned into a plasmid, which can be amplified in vitro via culturing of the host, isolated and purified, cleaved using methods and compositions of the present disclosure, and then subjected to further manipulation such as assembly. Furthermore, circular products (e.g., a plasmid) can also be produced by the methods and composition disclosed herein. For example, one or more of the construction oligonucleotides may be derived from a vector, such that when assembled, a full vector can be produced. The vector can then be transformed into a host cell (e.g., E. coli) for propagation.

Methods and compositions of the present disclosure can be used in the assembly of long-length polynucleotides (e.g., 10 kb or longer). In certain embodiments, small oligonucleotides (e.g., 100-800 bp or 500-800 bp) synthesized off of a chip can be first assembled into an intermediate polynucleotide, with or without using methods and compositions of the present disclosure. The intermediate polynucleotide can then be cloned into a plasmid, which can be introduced into a host, amplified via culturing, isolated and purified, cleaved using methods and compositions of the present disclosure, and then subjected to further assembly. This process can be repeated multiple times till the final long-length product is assembled.

In addition or as an alternative to direct ligation or polymerase assisted assembly, other methods can also be used to assemble cleavage products of the present disclosure. In some embodiments, the cleavage products can be subject to homologous recombination via SLiCE (Seamless Ligation Cloning Extract), as described in, for example, Zhang et al., Nucleic acids research 40.8 (2012): e55-e55 and U.S. Pub. No. 20130045508, incorporated herein by reference in their entirety. Briefly, SLiCE is a restriction site independent cloning/assembly method that is based on in vitro recombination between short regions of homologies (15-52 bp) in bacterial cell extracts derived from a RecA deficient bacterial strain engineered to contain an optimized λ prophage Red recombination system. Other recombination methods can also be used, such as recombination in yeast or phage. The cleavage products can be subject to Gibson assembly as described in, for example, Gibson et al., Nature Methods 6 (5): 343-345, and U.S. Pub. Nos. 20090275086 and 20100035768, incorporated herein by reference in their entirety. In Gibson assembly, DNA fragments containing ˜20-40 base pair overlap with adjacent DNA fragments are mixed with three enzymes, an exonuclease, a DNA polymerase, and a DNA ligase. In a one-tube reaction, the exonuclease creates overhangs so that adjacent DNA fragments can anneal, the DNA polymerase incorporates nucleotides to fill in any gaps, and the ligase covalently joins the DNA fragments.

As will be appreciated, the compositions and systems of the disclosure are useful in various areas of biotechnology, and particularly synthetic biotechnology, where site-directed nicking or cleaving of oligonucleotides is desired. For example, methods of the disclosure may be employed to cleave markers or selectable tags from nucleic acids. In one embodiment, the gRNA directs a fusion protein (e.g., fCas9) to a double stranded DNA sequence coding for an amino acid sequence selected from selectable marker(s) and/or tag(s) such as: ampicillin, kanamycin (KAN), tetracyclin (TET), glutathione-s-transferase (GST), maltose-binding protein (MBP), horse radish peroxidase (HRP), alkaline phosphatase (AP), red fluorescent protein (RFP), yellow fluorescent protein (YFP), green fluorescent protein (GFP), cyan fluorescent protein (CFP), FLAG, c-myc, human influenza hemaglutinin (HA), 6× histidine (6×His), and/or any combination thereof. In an embodiment, gRNA directs a fusion protein to a segment of a double stranded DNA that does not code for a selectable marker and/or tag.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for the use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. “Consisting essentially of” means inclusion of the items listed thereafter and which is open to unlisted items that do not materially affect the basic and novel properties of the invention.

### Example

A 2,000-mer is nicked at a first location using the fCas9 fusion protein bound to gRNA1 and at a second location using the fCas9 fusion protein bound to gRNA2. The resulting products are (1) a released Amp tag of 851 nucleotides (e.g., Amp sequence: ADA79624.1) and (2) a sequence of 1,149-mer, which contains an overhang. The 1,149-mer is bound to a bead at the non-overhang end and is then combined with a second sequence, which contains a complementary sticky end. A ligase is added to join the two sequences together on the bead. This additive process is continued until the desired polynucleotide length/sequence is synthesized. Then, the final product is cleaved from the bead and eluted. This process is sequential assembly.

Alternatively, multiple construction sequences can be nicked and/or cleaved in parallel to produce sticky ends that are pre-designed to be complementary to one another in a predetermined order, such that when combined in a ligation reaction, the construction sequences assemble in the predetermined arrangement.

Hierarchical assembly can also be used to produce the target product. For example, the construction sequences can be divided into two or more pools, each pool comprising a subsequence of the target product. After assembly of each pool of construction sequences into two or more subproducts, the subproducts can then be assembled into the final product.

The sequences below are non-limiting examples of the present disclosure:

SEQ ID NO.:1: Cas9

SEQ ID NO.:2: Cas9 nickase (D10A)

SEQ ID NO.:3: dCas9 (D10A and H840A); inactive Cas

SEQ ID NO.:4: FokI [partial amino acid sequence]

SEQ ID NO.:5: FokI (D69A) [partial amino acid sequence]

SEQ ID NO.:6: DNA coding sequence of wild-type Cas9 nuclease

SEQ ID NO.:7: DNA coding sequence of Cas9 nickase

SEQ ID NO.:8: DNA coding sequence of dCas9-NLS-GGS3linker-FokI

SEQ ID NO.:9: DNA coding sequence of NLS-dCas9-GGS3linker-FokI

SEQ ID NO.:10: DNA coding sequence of FokI-GGS3linker-dCas9-NLS

SEQ ID NO.:11: DNA coding sequence of NLS-FokI-GGS3linker-dCas9

SEQ ID NO.:12: DNA coding sequence of NLS-GGS-FokI-XTEN-dCas9 (“fCas9”)

SEQ ID NO.:13: (GGS)x3

SEQ ID NO.:14: FokI-(GGS)x6

SEQ ID NO.:15: FokI-L1

SEQ ID NO.:16: FokI-L2

SEQ ID NO.:17: FokI-L3

SEQ ID NO.:18: FokI-L4

SEQ ID NO.:19: FokI-L5

SEQ ID NO.:20: FokI-L6

SEQ ID NO.:21: FokI-L7

SEQ ID NO.:22: FokI-L8

SEQ ID NO.:23: FokI-L9

SEQ ID NO.:24: NLS-L3

### EQUIVALENTS

The present disclosure provides among other things novel methods and compositions for site-directed DNA nicking. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

The Sequence Listing filed as an ASCII text file via EFS-Web (file name: “014902PCT_ST25.txt”; date of creation: Jul. 7, 2015; size: 85,306 bytes) is hereby incorporated by reference in its entirety.

All publications, patents and sequence database entries mentioned herein are hereby incorporated by reference in their entireties as if each individual publication or patent was specifically and individually indicated to be incorporated by reference. In addition to all other publications, patents, and sequence database entries referenced and incorporated herein, reference is made to the following publications, each of which is also incorporated in its entirety herein:
Miller, et al., “An improved zinc-finger nuclease architecture for highly specific genome editing,” Nature Biotechnology, 25 (7), pp. 778-85 (2007)
Ramirez, et al., “Engineered zinc finger nickases induce homology-directed repair with reduced mutagenic effects,” Nucleic Acids Research, 40 (12), pp 5560-68 (2012)
Guilinger, et al., “Fusion of catalytically inactive Cas9 to FokI nuclease improves the specificity of genome modification,” Nature Biotechnology, 32 (6) pp. 577-83 (2014)
Tsia, et al, “Dimeric CRISPR RNA-guided FokI nucleases for highly specific genome editing,” Nature Biotechnology, 32 (6) pp. 569-77 (2014)
Mali, et al., “Cas9 as a versatile tool for engineering biology,” Nat Methods, 10 (10), pp. 957-63 (2013)
Bassett, et al., “Highly Efficient Targeted Mutagenesis of Drosophila with CRISPR/Cas9 System,” Cell Reports 4, pp. 220-28 (2013)
Christian, et al., “Targeting DNA Double-Strand Breaks with TAL Effector Nucleases,” Genetics 186, pp. 757-61 (2010)
Lippow, et al., “Creation of a type IIS restriction endonuclease with a long recognition sequence,” Nucleic Acids Research, 37 (9), pp. 3061-73 (2009)
Looney, et al., “Nucleotide sequence of the FokI restriction-modification system: separate strand-specificity domains in the methyltransferase,” Gene, 80 (2), pp. 193-208
Jacobson, et al., “Methods and Devices for Nucleic Acid Synthesis,” U.S. Patent Application Publication No. 2013/0296294
Kung, et al., “Methods for Preparative In Vitro Cloning,” International Patent Application Publication No. WO2012/174337
Jacobson, et al., “Compositions and Methods for High Fidelity Assembly of Nucleic Acids,” International Patent Application Publication No. WO2013/032850
Jacobson, et al., “Methods for Nucleic Acid Assembly and High Throughput Sequencing,” International Patent Application Publication No. WO2014/004393
Kung, et al., “Methods for Sorting Nucleic Acids and Muliplexed Preparative In Vitro Cloning,” International Patent Application Publication No. WO2013/163263
Joung, et al., “TALENs: a widely applicable technology for targeted genome editing,” Nat. Rev. Mol. Cell Biol. 14, 49-55 (2012).
Urnov, et al., “Genome editing with engineered zinc finger nucleases,” Nat. Rev. Genet. 11, 636-646 (2010).
Silva et al., “Meganucleases and Other Tools for Targeted Genome Engineering: Perspectives and Challenges for Gene Therapy,” Curr Gene Ther. February 2011; 11(1): 11-27.

1. A method for assembling a target nucleic acid, the method comprising:
(a) providing a plurality of double-stranded polynucleotides, each polynucleotide comprising a flanking region on the 3′ end, or 5′ end, or 3′ end and 5′ end, wherein the flanking region comprises a first recognition site and a second recognition site, wherein the plurality of double-stranded polynucleotides together comprise the target nucleic acid;
(b) nicking, in vitro, a first strand of each polynucleotide with a first nickase to produce a first nick, wherein the first nickase is configured to recognize and bind to the first recognition site; and
(c) nicking, in vitro, a second strand of each polynucleotide with a second nickase to produce a second nick, wherein the second nickase is configured to recognize and bind to the second recognition site, thereby producing cleaved polynucleotide fragments, each having an overhang defined by the first nick and the second nick, wherein the overhang is predesigned by selecting the first and second recognition sites and
(d) assembling the cleaved polynucleotide fragments at the overhangs to form the target nucleic acid, wherein a first overhang of a first cleaved polynucleotide fragment is complementary to a second overhang of a second cleaved polynucleotide fragment, and wherein said assembling comprises ligating the assembled cleaved polynucleotide fragments to produce a ligated product,
wherein at least one of the plurality of double-stranded polynucleotides was provided on a solid support.

2. The method of claim 1, wherein the first nickase or the second nickase comprises one or more of: Cas9 fused to a nuclease via a linker at the N terminus (“fCas9”), Cas9 fused to a nuclease via a linker at the C terminus (“Cas9f”), RISC complexed with or fused to a nuclease, transcription activator-like effector (TALE) complexed with or fused to a nuclease, zinc-finger complexed with or fused to a nuclease, meganuclease, and any combination thereof.

3. The method of claim 2, wherein the Cas9 is catalytically inactive and/or the nuclease is incapable of binding to DNA.

4. The method of claim 2, wherein the nuclease is FokI.

5. The method of claim 4, wherein the FokI is a catalytically inactive monomer of FokI cleavage domain.

6. (canceled)

7. The method of claim 4, wherein the first nickase or the second nickase is a dimer wherein the FokI dimerizes with a catalytically active monomer of FokI cleavage domain.

8. The method of claim 4, wherein the FokI is a catalytically active monomer of FokI cleavage domain.

9. The method of claim 8, wherein the first nickase or the second nickase is a dimer wherein the FokI dimerizes with a catalytically active or inactive monomer of FokI cleavage domain.

10. The method of claim 7, wherein the first nickase or the second nickase is a heterodimer.

11. The method of claim 2, wherein in the first nickase, the Cas9 or RISC is directed by a first guide sequence such as gRNA to the first site, wherein the first guide sequence comprises a first sequence that is complementary to the first site.

12. The method of claim 11, wherein in the second nickase, the Cas9 or RISC is directed by a second guide sequence such as gRNA to the second site, wherein the second guide sequence comprises a second sequence that is complementary to the second site.

13. The method of claim 12, wherein the first guide sequence and the second guide sequence are non-naturally occurring.

14. The method of claim 12, wherein the first nickase and the second nickase nick at a predetermined position upstream or downstream to the first site and the second site, respectively, to produce the first nick and the second nick, respectively.

15. The method of claim 14, wherein the first and second sites are selected such that the first nick and the second nick are offset by a predefined number of nucleotides.

16-38. (canceled)

39. The method of claim 2, wherein the nuclease is FokI.

40. The method of claim 9, wherein the first nickase or the second nickase is a heterodimer.

41. The method of claim 39, wherein the FokI is a catalytically inactive monomer of FokI cleavage domain.

42. The method of claim 39, wherein the FokI is a catalytically active monomer of FokI cleavage domain.


SEQUENCE LISTING (25 sequences)

SEQ ID NO: 1 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1368 aa, Unknown; identical to SEQ ID NO: 1
  Mutant

SEQ ID NO: 3 - protein, 1368 aa, Unknown
  Mutant
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 198 aa, Flavobacterium okeanokoites
GSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 5 - protein, 198 aa, Unknown
  Mutant
GSQLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 6 - DNA, 4212 bp, Streptococcus pyogenes
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 7 - DNA, 4221 bp, Unknown
  Mutant
atggactataaggaccacgacggagactacaaggatcatgatattgattacaaagacgatgacgataagatggccccaaagaagaagcggaaggtcggtatccacggagtcccagcagccgataaaaagtattctattggtttagctatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgac

SEQ ID NO: 8 - DNA, 4834 bp, Unknown
  Synthetic
atggataaaaagtattctattggtttagctatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggcgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaaaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcaggatcaggtggaagtggcggcagcggaggttctggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaacttt

SEQ ID NO: 9 - DNA, 4845 bp, Unknown
  Synthetic
atggactacaaagaccatgacggtgattataaagatcatgacatcgattacaaggatgacgatgacaagatggcccccaagaagaagaggaaggtgggcattcaccgcggggtacctatggataaaaagtattctattggtttagctatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactcaggtggaagtggcggcagcggaggttctggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaacttt

SEQ ID NO: 10 - DNA, 4836 bp, Unknown
  Synthetic
atgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttggcggtagtgggggatctgggggaagtatggataaaaagtattctattggtttagctatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 11 - DNA, 4854 bp, Unknown
  Synthetic
atggactacaaagaccatgacggtgattataaagatcatgacatcgattacaaggatgacgatgacaagatggcccccaagaagaagaggaaggtgggcattcaccgcggggtacctggaggttctatgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttggcggtagtgggggatctgggggaagtatggataaaaagtattctattggtttagctatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgac

SEQ ID NO: 12 - DNA, 4875 bp, Unknown
  Synthetic
atggactacaaagaccatgacggtgattataaagatcatgacatcgattacaaggatgacgatgacaagatggcccccaagaagaagaggaaggtgggcattcaccgcggggtacctggaggttctatgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttagcggcagcgagactcccgggacctcagagtccgccacacccgaaagtatggataaaaagtattctattggtttagctatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgac

SEQ ID NO: 13 - protein, 9 aa, Unknown
  Synthetic
GGSGGSGGS

SEQ ID NO: 14 - protein, 18 aa, Unknown
  Synthetic
GGSGGSGGSGGSGGSGGS

SEQ ID NO: 15 - protein, 10 aa, Unknown
  Synthetic
MKIIEQLPSA

SEQ ID NO: 16 - protein, 10 aa, Unknown
  Synthetic
VRHKLKRVGS

SEQ ID NO: 17 - protein, 15 aa, Unknown
  Synthetic
VPFLLEPDNINGKTC

SEQ ID NO: 18 - protein, 12 aa, Unknown
  Synthetic
GHGTGSTGSGSS

SEQ ID NO: 19 - protein, 7 aa, Unknown
  Synthetic
MSRPDPA

SEQ ID NO: 20 - protein, 12 aa, Unknown
  Synthetic
GSAGSAAGSGEF

SEQ ID NO: 21 - protein, 12 aa, Unknown
  Synthetic
SGSETPGTSESA

SEQ ID NO: 22 - protein, 16 aa, Unknown
  synthetic
SGSETPGTSESATPES

SEQ ID NO: 23 - protein, 21 aa, Unknown
  synthetic
SGSETPGTSESATPEGGSGGS

SEQ ID NO: 24 - protein, 12 aa, Unknown
  synthetic
SIVAQLSRPDPA

SEQ ID NO: 25 - protein, 4 aa, Unknown
  synthetic
GGSM